(19) United States
(12) Patent Application Publication
(10) Pub. No.: US 2026/0204376 A1
(43) Pub. Date: Jul. 16, 2026

(54) INFORMATION PROCESSING METHOD, PROGRAM AND INFORMATION PROCESSING APPARATUS

(71) Applicants: VIE, INC., Kamakura-shi, Kanagawa (JP); NATIONAL CANCER CENTER, Chuo-ku, Tokyo (JP)

(72) Inventors: Takuya IBARAKI, Kamakura-shi, Kanagawa (JP); Tomonori YANO, Chuo-ku, Tokyo (JP); Yusuke YODA, Chuo-ku, Tokyo (JP); Hironori SUNAKAWA, Chuo-ku, Tokyo (JP); Takashi WATANABE, Chuo-ku, Tokyo (JP); Manabu HASHIMOTO, Chuo-ku, Tokyo (JP)

(21) Appl. No.: 19/127,994

(22) PCT Filed: Nov. 8, 2023
(86) PCT No.: PCT/JP2023/040207

§ 371 Date: May 7, 2025

(30) Foreign Application Priority Data
Nov. 8, 2022 (JP) 2022-178986

(51) Int. Cl. G16H 20/10; A61B 5/00; A61B 5/16; A61B 5/372

(52) U.S. Cl. CPC G16H 20/10; A61B 5/16; A61B 5/372; A61B 5/6803

(57) ABSTRACT

Provided is an information processing method executed by an information processing apparatus, the method including: sequentially acquiring brainwave signals from a brainwave measuring device attached to ears of a user; predicting sedation depth with respect to the sequentially acquired brainwave signals by using the sequentially acquired brainwave signals, and a learning model which has learned learning data including brainwave signals and sedation depth with respect to the brainwave signals; and outputting sedation depth information on the predicted sedation depth.

### TECHNICAL FIELD

The present invention relates to an information processing method, a program, and an information processing apparatus.

### BACKGROUND ART

As a method for ascertaining a sedated state of an anesthetized patient, a bispectral index (BIS) technique that monitors brainwaves is known. Besides BIS, a technique to apply electric stimulation to a living body and ascertain a sedation state based on a reaction of the living body, and to output an index related to the sedation of the patient based on the ascertained state, is also known (e.g. see Patent Literature 1). Further, a technique to estimate a sedation level from a brainwave signals using machine learning is also available (e.g. see Patent Literature 2).

### CITATION LIST

### Patent Literature

Patent Literature 1: Patent Publication JP-A- 2017-148183

Patent Literature 2: US Patent No. 2020/0253544

### SUMMARY OF INVENTION

### Technical Problem

The monitoring technique of BIS, which can evaluate the sedation depth without applying electric stimulation to the living body, is widely used in the field of general anesthesia, but this technique has various problems. For example, it is reported that accuracy and reliability are low in the moderate sedation level, and due to the micro-projections on the surface of electrodes, pain is experienced when attaching electrodes to a patient in the conscious state, and contact dermatitis tends to occur (the same in the case of Patent Literature 1). In the case of Patent Literature 2, a sensor which includes at least six electrodes and which is adhered to the skin of the forehead (e.g. SedLine (registered trademark) from Mashimo Corp.) is used, hence there is a concern about discomfort and inconvenience of attaching the sensor. Evaluation of sedation depth is demanded not only for anesthesia, but also for sleeping pills and tranquilizers.

With the foregoing in view, an object of an aspect of the disclosed technique is to provide an information processing method, a program, and an information processing apparatus which implements a convenient and appropriate sedation monitoring method.

### Solution to Problem

In an information processing method according to aspects of the disclosed technique, an information processing apparatus executes: sequentially acquiring brainwave signals from a brainwave measuring device attached to ears of a user; predicting sedation depth with respect to the sequentially acquired brainwave signals by using the sequentially acquired brainwave signals and a learning model which has learned learning data including brainwave signals and sedation depth with respect to the brainwave signals; and outputting sedation depth information on the predicted sedation depth.

### Advantageous Effect of Invention

According to an aspect of the disclosed technique, a convenient and appropriate sedation monitoring method can be implemented.

### BRIEF DESCRIPTION OF DRAWINGS

FIG. 1 is a schematic diagram depicting an example of an information processing system according to an embodiment.

FIG. 2 is a diagram depicting an example of an earphone set according to an embodiment.

FIG. 3 is a schematic cross-sectional view of an example of an earphone according to an embodiment.

FIG. 4 is a diagram depicting an application example of an information processing system 1 according to an embodiment.

FIG. 5 is a table indicating scores of a sedation scale of RASS.

FIG. 6 is a diagram indicating an example of data on a subject A in an experiment.

FIG. 7 is a diagram indicating classification accuracies of the subject A.

FIG. 8 is a graph indicating a relationship of RASS, BIS and a prediction model of the subject A during the entire measurement period.

FIG. 9 is a diagram indicating a relationship of three signals of brainwave signals and sedation depths.

FIG. 10 is a table indicating accuracy of predicted values of the prediction model of each subject.

FIG. 11 is a graph indicating a result of a t-test using correlation coefficients.

FIG. 12 is a bock diagram depicting an example of a server according to an embodiment.

FIG. 13 is a block diagram depicting an example of a processing terminal according to an embodiment.

FIG. 14 is a flow chart depicting an example of prediction model generating processing by a server according to an embodiment.

FIG. 15 is a flow chart depicting an example of prediction processing of sedation depth by a processing terminal according to an embodiment.

FIG. 16 is a flow chart depicting an example of learning model generating processing by a processing terminal according to an embodiment.

FIG. 17 is a diagram indicating an example of a display screen according to an embodiment.

### DESCRIPTION OF EMBODIMENTS

Embodiments of the present invention will now be described with reference to the drawings. The embodiments to be described below are examples, and are not intended to exclude the application of various modifications and techniques that are not explicitly indicated below. In other words, the present invention may be modified in various ways within a scope not departing from the spirit thereof. In the following description of the drawings, same or similar portions are denoted with a same or similar reference sign. The drawings are schematically expressed, of which dimensions, ratios and the like do not always match with actual values. Among the drawings as well, dimensional relationships and ratios of each portion may not always be consistent.

### Embodiments

An overview of a system according to an embodiment will now be described with reference to the drawings.

### Overview of System

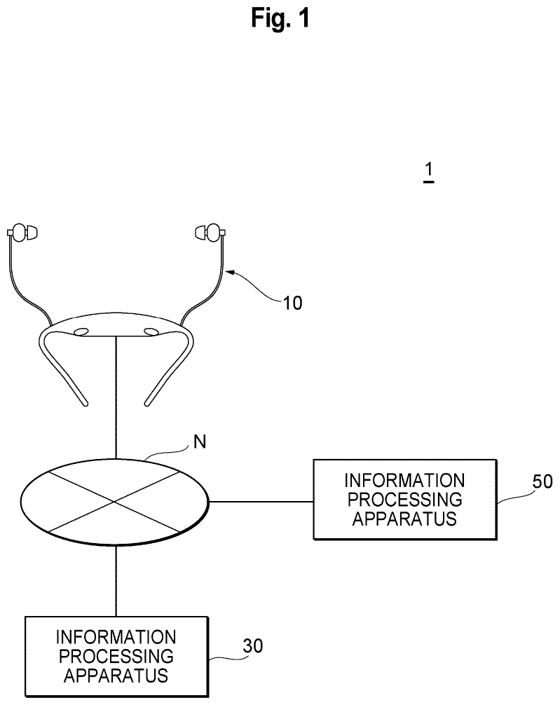

An example of an information processing system 1 according to an embodiment will be described first with reference to FIG. 1. In the information processing system 1, a user, of which brainwaves (Electroencephalogram:EEG) will be measured, wears an earphone set 10 as a brainwave measuring device, where bioelectrodes are disposed in ear canals.

In the example illustrated in FIG. 1, a neck suspension type earphone set 10 is illustrated, but any earphones may be used as long as brainwave signals can be sensed through the ear canals. For example, an earphone set which can acquire reference signals through ear lobes, an earphone that acquires reference signals and ground signals from other positions (position other than ear canals), completely wireless earphones and the like, can be used. The brainwave measuring device is not limited to the earphone type, and may be a headphone type where bioelectrodes are disposed in earmuff portions, for example.

In the case of the example illustrated in FIG. 1, the earphone set 10 acquires brainwave signals through the ear canals, and sends the brainwave signals to an information processing apparatus 30 or to an information processing apparatus 50 via a network N. The network N includes a cable or wireless network, and for which a short range wireless communication, such as Bluetooth (registered trademark) may be used.

The earphone set 10 may execute predetermined processing on the brainwave signals and send the processed signals to the information processing apparatus 30 which plays a role of a server, or to the information processing apparatus 50 which the user is using. The predetermined processing includes at least one of: amplifying processing, sampling, filtering and difference calculation, and the like.

The information processing apparatus 30 is a server, for example, and acquires brainwave signals to be measured by the brainwave measuring device, and executes each processing. For example, the information processing apparatus 30 extracts feature data from the brainwave signals of the user (e.g. patient) to whom anesthetic is being administered, and performs machine learning using learning data, which is generated by labeling the sedation depth of the user on this feature data, whereby a prediction model to predict a sedation depth is generated. This makes it possible to generate a prediction model to predict the sedation depth by acquiring brainwave signals using a simple device, while minimizing the burden on the user when attaching this device.

The information processing apparatus 30 may send the generated prediction model to the information processing apparatus 50. Further, the information processing apparatus 50 may acquire brainwave signals first, and the information processing apparatus 30 may acquire the brainwave signals via a predetermined application. Furthermore, the information processing apparatus 30 may calculate feature data based on the sequentially acquired brainwave signals, input this feature data to the prediction model, and send sedation depth information, including the predicted sedation depth, to the information processing apparatus 50.

The information processing apparatus 50 is a processing terminal, such as a portable terminal held by the user, for example, and sequentially acquires brainwave signals from the earphone set 10. The information processing apparatus 50 may input the sequentially acquired brainwave signals to the prediction model and acquire the sedation depth information thereby. The information processing apparatus 50 may also output the acquired brainwave signals or feature data to the information processing apparatus 30, and acquire the sedation depth information from the information processing apparatus 30.

Based on the predicted separation depth, the information processing apparatus 50 may output an alarm, a predicted dosage of anesthetic, a waveform of a sedation depth, and the like on the display, or may output sound data and the like to a sound output apparatus (e.g. earphone set 10). Thereby information on the sedation depth predicted from the brainwave signals can be outputted, and the sedation state of the patient can be notified to other users (e.g. physicians).

### Configuration of Earphone Set

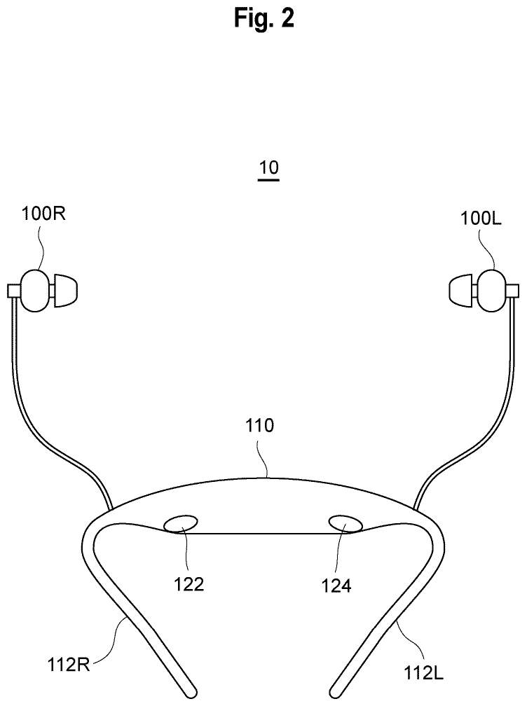

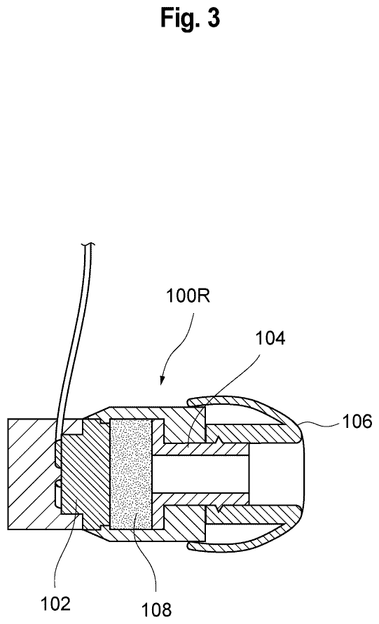

An overview of the earphone set 10 according to an embodiment will be described with reference to FIGS. 2 and 3. The earphone set 10, however, is not limited to the example illustrated in FIGS. 2 and 3, and the technique of the present disclosure is applicable to any earphones as long as brainwaves can be sensed through ear canals and outputted to an external device.

FIG. 2 is a diagram depicting an example of the earphone set 10 according to an embodiment. The earphone set 10 illustrated in FIG. 2 includes a pair of earphones 100L and 100R, and a neck suspending portion 110. Each earphone 100L and 100R is connected to the neck suspending portion 110 via a cable that is communicable with signals, but may be connected via wireless communication. Hereafter L and R will be omitted unless distinction between left and right is necessary.

The neck suspending portion 110 includes a center member that is placed along the back of the neck, and bar members (arms) 112L and 112R which are curved along both sides of the neck. On a surface of the center member which contacts with the neck on the back side, electrodes 122 and 124 are disposed to sense brainwave signals. The electrodes 122 and 124 are an electrode for ground connection and a reference electrode respectively. Thereby, as mentioned later, distance can be taken from elastic electrodes disposed on ear chips of the earphones, and brainwave signals can be acquired accurately. The neck suspending portion 110 may include a processing unit to process the brainwave signals and a communication device to communicate with external units, but the processing unit and the communication unit may be disposed on the earphones 100.

The front end side of the bar members 112L and 112R on both sides of the neck suspending portion 110 is heavier than the root side (center member side) thereof, so that the electrodes 122 and 124 are appropriately pressed on the neck of the user. For example, weights are disposed on the front end side of the bar members 112L and 112R. The positions of the electrodes 122 and 124 are not limited to the illustrated positions.

FIG. 3 is a schematic cross-sectional view of an example of the earphone 100R according to an embodiment. For the earphone 100R illustrated in FIG. 3, an elastic member (e.g. urethane) 108 may be disposed between a speaker 102 and a nozzle 104. If the elastic member 108 is disposed, vibration of the speaker 102 is less likely to transfer to the elastic electrode of an ear chip 106, and sound interference between the elastic electrode of the ear chip 106 and the speaker 102 can be prevented.

The ear chip 106, including the elastic electrode, is located at a sound guiding opening, and can prevent interference caused by sound vibration using the elasticity of the elastic electrode. An elastic member is also used for housing, thereby the sound vibration is less likely to transfer to the elastic electrode of the ear chip 106, which can prevent interference caused by sound vibration.

The earphone 100 may include an audio sound processor, and sound signals at a predetermined frequency (e.g. 50 Hz) or less, which correspond to brainwave signals, may be cutoff using this audio sound processor. The audio sound processor cuts off the sound signals at 30 Hz or less in particular, which is the frequency band where features of brainwave signals appear more clearly, but may amplify sound signals at a frequency around 70 Hz, so that the base sound is not diminished.

Thereby interference of the sound signals and brainwave signals can be prevented. The audio sound processor may cutoff a predetermined frequency only when brainwave signals are being sensed.

The ear chip 106 transfers brainwave signals sensed through the ear canal to the contact of the electrode disposed on the nozzle 104. The brainwave signals are transferred from the ear chip 106 to a biosensor (not illustrated) inside the earphone 100 via the contact. The biosensor outputs the sequentially acquired brainwave signals to a processing apparatus disposed on the neck suspending portion 110 via a cable, or transmits the brainwave signals to an external device. The ear chip 106 and the housing, which includes the biosensor and the audio sound processor, may be insulated from each other.

### Application Example of System

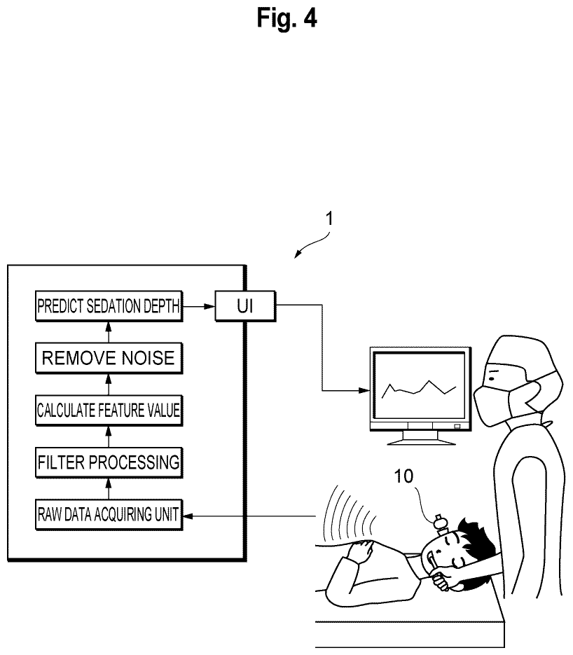

FIG. 4 is a diagram depicting an application example of the information processing system 1 according to an embodiment. In the example illustrated in FIG. 4, the earphone set 10 is attached on a patient, and an agent (e.g. anesthetic) is administered before surgery. Here the brainwave signals of the user are acquired via the electrodes of the earphone set 10.

Filter processing, feature value calculation, noise removal, and the like are performed on the acquired brainwave signals, and sedation depth is predicted from the brainwave signals after removing noise. The predicted sedation depth may be outputted to a medical device and the like via a user interface. To predict the sedation depth of the user, machine learning is performed based on the brainwave signals of the user and the sedation depth linked to the brainwave signals. Thereby a prediction model, to predict the sedation depth from the brainwave signals, is constructed. In the following, experiments conducted by the inventors to construct the prediction model in the information processing system of the present disclosure will be described.

### Overview of Experiment

### Subject

The subjects of the present experiment are 10 patients, for which endoscopic treatment of esophageal cancer is performed.

### Acquired Items

Using a bispectral index (BIS) monitoring system made by Nihon Kohden Corp., a heart rate (HR), a pulse rate (PR) and a respiratory rate (RR) per minute, an arterial oxygen saturation (SpO2) measured by a pulse oximeter, BIS, and SQIBIS (value of signal quality of BIS) are acquired. Further, using the earphone set 10 illustrated in FIGS. 1 to 3, brainwave signals are acquired through the ear canals of both ears at a 600 Hz sampling rate. In the case of the earphone set 10, the reference electrode and the ground electrode are disposed on the back of the neck (electrodes 122 and 124 in FIG. 2). Signals from the left ear and the right ear and a difference signal between the left and right ears, a total of three signals, are used. The difference signal is used as a signal after removing noise commonly mixed in the left and right electrodes.

### Acquiring Method

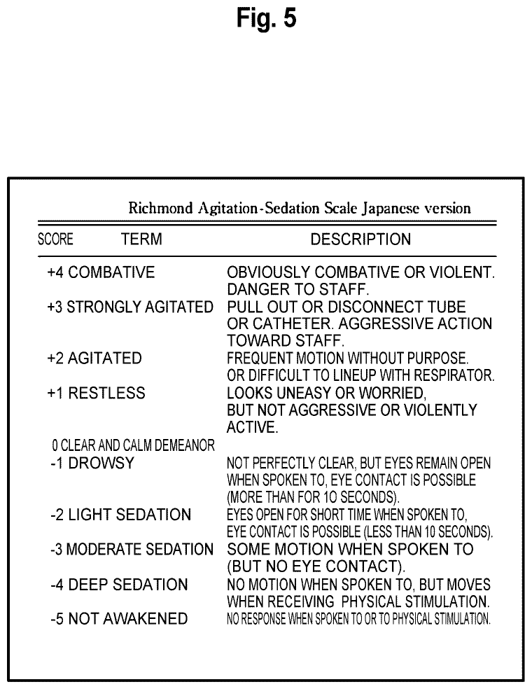

Before sedating the subject, various sensors are attached to the subject and recording starts. RASS (described later with reference to FIG. 5) is manually evaluated at 1 to 10 minute intervals. Data on agents and the like is also inputted from the system and recorded. In this experiment, to determine RASS, physical stimulation (e.g. hitting, pinching) is applied to the legs, and patient reaction is recorded since it is not possible to speak to the patient or perform chest compression. Such an event as body movement is also manually recorded. BIS data and brainwave data are synchronized at the recording timing of a personal computer (PC).

### Processing

For the earphone set 10, 1 to 40 Hz band pass filters are used for all three signals. From each electrode of the earphone set 10, data is extracted with a 4 second time window and a 1 second slide. 1 to 40 Hz power (Welch's power spectral density) is calculated at 0.5 Hz intervals. If the average of the absolute amplitude values of each window data exceeds 50 μV (threshold) in at least one of the three signals, these signals are removed as noise. The power in each frequency band is standardized for the subject, and principal component analysis is performed. 1 to 100 dimensional data are extracted and are further standardized.

### Teaching Label

FIG. 5 is a table indicating the scores of a sedation scale of the Richmond Agitation-Sedation Scale (RASS). As indicated in FIG. 5, RASS is expressed as a score from −5 to +4. In this experiment, in order to generate teacher data of the sedation depth prediction model (hereafter also called “prediction model”), the score of RASS is classified into the following teaching labels. In this experiment, the labeling is performed with a moderate sedation as the boundary. For example, an index that increases as the subject approaches the awakened state (arousal state) is used just like RASS, and the index equal to or more than the moderate sedation means a state from “moderate sedation” to “combative”, and the index less than the moderate sedation means “deep sedation” or “not awakened”.
Teaching label 1: state closer to awakening, equal to or more than moderate sedation of which RASS score is −3 or more
Teaching label 0: state of deep sedation, less than moderate sedation of which RASS score is −4 or less

### Constructing Prediction Model

Out of the brainwave data acquired from each subject, resampling is performed randomly from the larger number of data, in accordance with the smaller number of data. For example, if a number of awakened data is 100 and a number of deep sedation data is 500, 100 data is randomly sampled from the data of deep sedation. Out of this sampled data, 90% is randomly sampled as learning data (training data), and 10% is randomly sampled as test data. As the learning model, a sparce modeling of a generalized linear model, that is a least absolute shrinkage and selection operator (LASSO), is used for the learning data, and 10-division cross-validation is performed for 20λ parameters, so as to determine λ to be a minimum error. The above mentioned prediction model may be created for each individual subject.

### Accuracy Index

In this experiment, the following two indices are used.
Index 1: accuracy based on binary classification in test data (same number for positive and negative)
Index 2: correlation coefficient of RASS, prediction model and BIS for all data (max=1)

For the predicted values in a time series, in a 0 to 1 likelihood, the RASS value is calculated by 0-(1—predicted value)×5, and the data movement average value for the most recent 10 seconds is used (purpose of smoothing).

### Example of Subject A

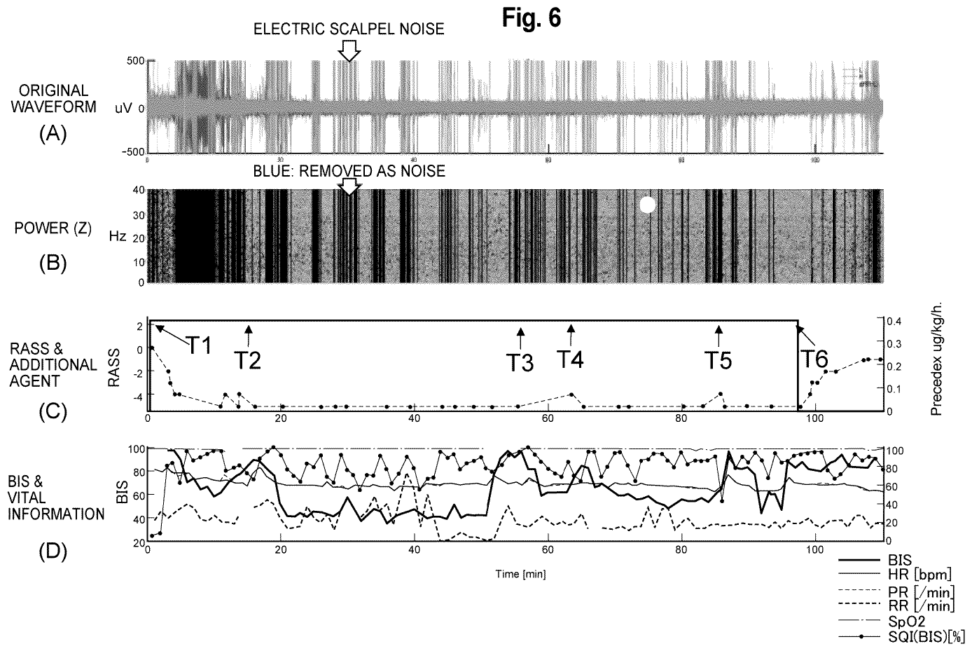

An example of data on a subject A in the present experiment will be described next. FIG. 6 is a diagram indicating an example of data on the subject A in the present experiment. FIG. 6(A) is a diagram indicating an original waveform of the subject A, who was measured using the earphone set 10. The ordinate indicates an amplitude value (unit: μV), and the abscissa indicates measurement time (unit: minutes). In FIG. 6(A), the original forms of the three signals (left, right, difference between left and right) are indicated. As the example in FIG. 6(A) indicates, a noise appears every time an electric scalpel is used during surgery.

FIG. 6(B) is a diagram indicating a spectral power (Z) of a differential signal. The ordinate indicates a spectral power value (unit: Hz), and the abscissa indicates measurement time. A Z value is expressed by a value in the 1 to 40 Hz range, as mentioned above. As indicated in FIG. 6(B), when the electric scalpel is used, [the spectral power value is specified as noise, and is not used as learning data.

FIG. 6(C) is a diagram indicating an example of a relationship between RASS and an additional agent. The left ordinate indicates a score of RASS, the right ordinate indicates a dosage of the agent, and the abscissa indicates the measurement time. The example in FIG. 6(C) indicates that an anesthetic was administered at timings T1 to T5, and an antagonist was administered at timing T6. The timings T2, T4 and T5 are timings when RASS indicates −4, and T4 and T5 are timings when body movement was observed.

FIG. 6(D) is a diagram indicating an example of a relationship between BIS and vital information. The left ordinate indicates a score of BIS, the right ordinate indicates SQIBIS (unit: %), and the abscissa indicates the measurement time. In the example in FIG. 6(D), BIS, HR, PR, RR, SpO2 and SQIBIS are measured and indicated in one graph.

### Classification Accuracy of Subject A

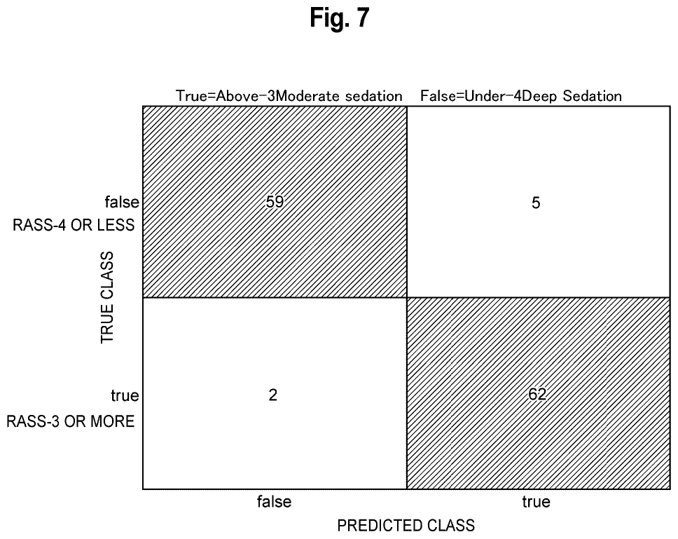

FIG. 7 is a diagram indicating a classification accuracy of the subject A. As the example in FIG. 7 indicates, a value predicted from brainwave signals using a prediction model based on the above mentioned sparse modeling LASSO and actual RASS observed visually are compared. Using the brainwave signals of the subject A, where RASS—3 or more is classified as moderate sedation or more and RASS—4 or less is classified as less than moderate sedation, then the classification accuracy in this case is 94.5312%. This indicates the brainwave signals acquired by the earphone set 10 is at a practical level.

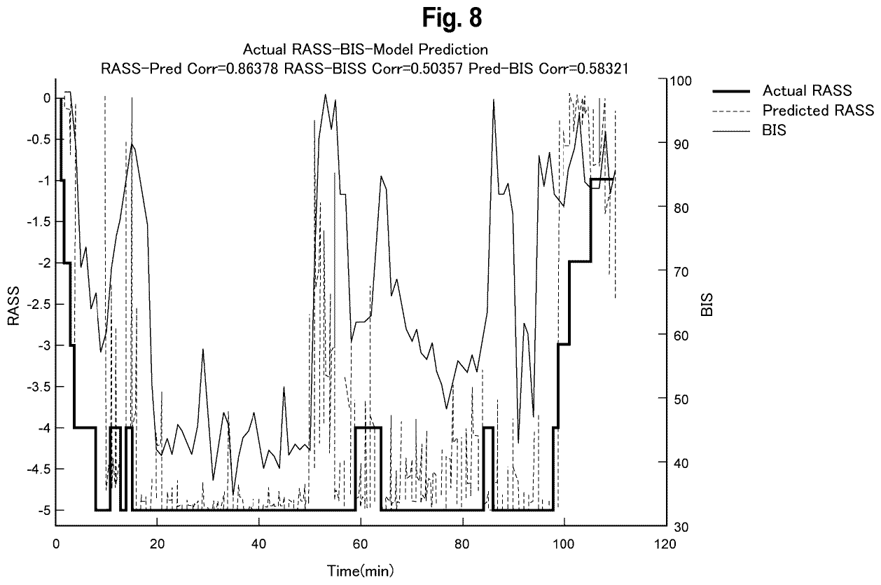

FIG. 8 is a graph indicating a relationship between RASS, BIS and a predicted value of a prediction model of the subject A during the entire measurement period. In FIG. 8, the left ordinate indicates the value of RASS, the right ordinate indicates the value of BIS, and the abscissa indicates the measurement time. Various conditions and results of the correlation coefficients are as follows.

### Various Conditions

For the value of RASS, a nearest neighbor value of the target brainwave time window is used.

For the predicted value, binary classification (0 or 1) is normally used, but to make comparison easier, 0-(predicted value)×5 is used.

For the brainwave signals, a movement average filter is used for data of the most recent 10 seconds.

### Result of Correlation Coefficients

Correlation coefficient between RASS and predicted value: 0.86378
Correlation coefficient between RASS and BIS: 0.50357
Correlation coefficient between predicted value and BIS: 0.58321

The example in FIG. 8 indicates that the correlation between the actual value of RASS and the predicted value of RASS is high.

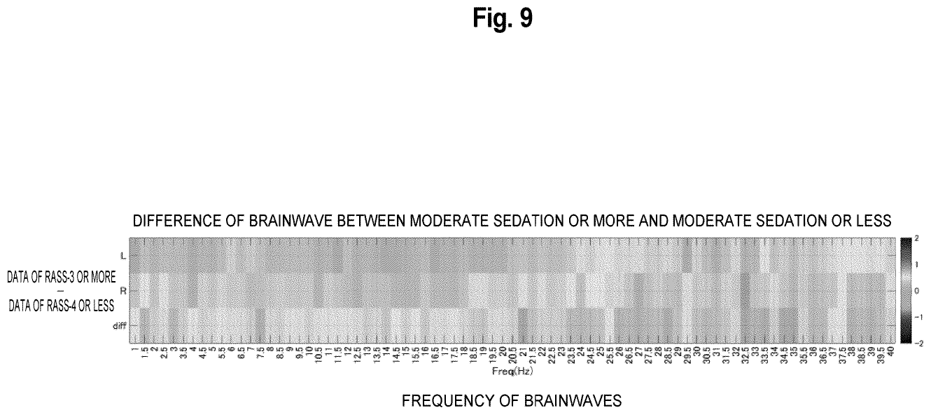

FIG. 9 is a diagram indicating a relationship of the three signals of brainwave signals and sedation depths. The ordinate of the graph in FIG. 9 indicates a power value (Z) of the brainwaves determined by subtracting data of RASS—4 or less from data of RASS—3 or more, and the abscissa indicates the frequency of brainwaves. Out of the three signals, the differential signal more easily generates the difference of power at each frequency, and the difference of power is more easily generated as the frequency becomes higher.

### Accuracy of Predicted Values of Each Subject

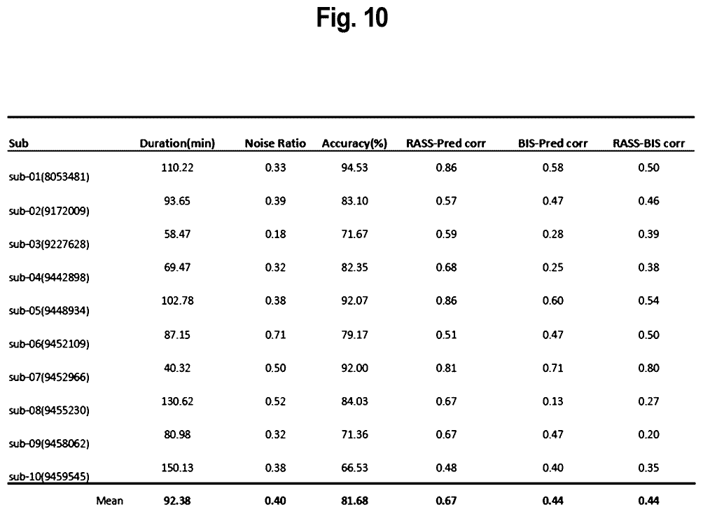

FIG. 10 is a table indicating accuracy of predicted values of the prediction model of each subject. As mentioned above, the accuracy of the prediction model of the subject A, indicated in FIG. 10, is 94.53%, and the accuracies of the other 9 subjects are also in the 66.53% to 92.07% range, and the average value thereof is 81.68%.

A first average value of the correlation coefficient between RASS and the predicted value is 0.67, a second average value of the correlation coefficient between BIS and the predicted value is 0.44, and a third average value of the correlation coefficient between RASS and BIS is 0.44. In other words, the first average value is larger than the third average value, which means that correlation with RASS is higher than the correlation with BIS.

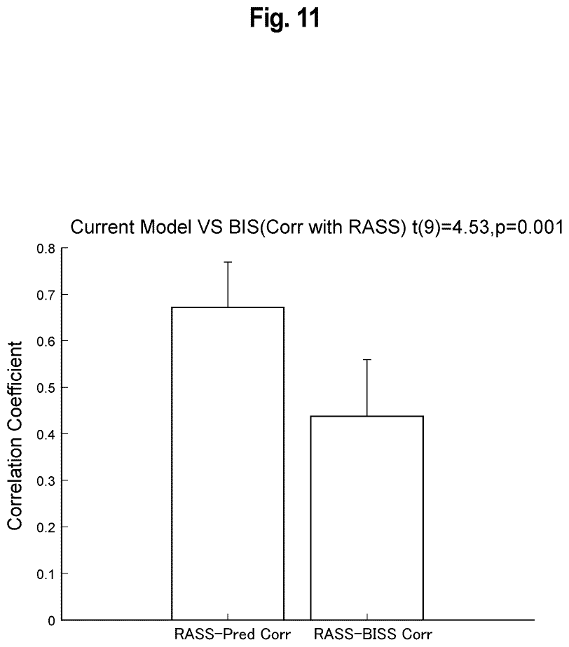

FIG. 11 is a graph indicating a result of a t-test using correlation coefficients. In the example in FIG. 11, the left graph indicates the correlation coefficient between RASS and the predicted value, and the right graph indicates the correlation coefficient between RASS and BIS. As indicated in FIG. 11, the t value is t(9)=4.53, indicating that there is a significant difference in the average value, and the p value is p=0.001, indicating that this t value is not a coincidence. Therefore according to this experiment result, brainwave signals can be measured using the earphone set 10 at clinically usable accuracy, whereby sedation depth can be appropriately predicted. Based on this experiment result, the configuration of each information processing apparatus will be described.

### Configuration Example of Server

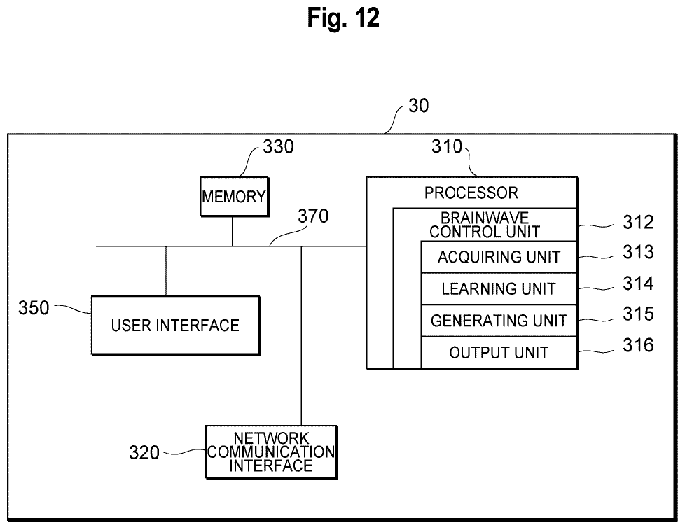

FIG. 12 is a block diagram depicting an example of the information processing apparatus 30. The information processing apparatus 30 is a server, for example, and may be constituted of one or a plurality of apparatuses. The information processing apparatus 30 processes brainwave signals or brainwave information, and performs processing to predict the sedation depth from the brainwave signals using a learning function of artificial intelligence, for example. The information processing apparatus 30 is also called a server 30. The information processing apparatus 30 need not always be a server, and may be a general purpose computer.

The server 30 includes: one or a plurality of processors (central processing units: CPUs), 310, one or a plurality of network communication interfaces 320, a memory 330, a user interface 350, and one or a plurality of communication buses 370 to interconnect these composing elements.

In some cases the server 30 may include the user interface 350, for example, such as a display device (not illustrated), and a keyboard and/or a mouse (or some input device, such as a pointing device (not illustrated)).

The memory 330 is, for example, DRAM, SRAM, DDR RAM or other high-speed random access memories, such as a random access solid-state storage device. The memory 330 may also be one or a plurality of magnetic disk storage devices, optical disk storage devices, flash memory devices, or other non-volatile memories, such as non-volatile solid-state storage devices. The memory 330 may also be a computer-readable non-transitory recording medium in which programs are recorded.

Another example of the memory 330 is one or a plurality of storage devices which are remotely installed from the processor 310. In some cases, the memory 330 stores the following programs, modules, data structures or subsets thereof.

One or a plurality of processors 310 read programs from the memory 330 as required, and executes those programs. For example, the one or plurality of processors 310 may implement a brainwave control unit 312, an acquiring unit 313, a learning unit 314, a generating unit 315 and an output unit 316 by executing the programs stored in the memory 330. The brainwave control unit 312 controls or processes the sequentially acquired brainwave signals, and controls the following processing.

The acquiring unit 313 acquires brainwave signals measured by the bioelectrodes included in the brainwave measuring device, such as the earphone set 10. For example, the acquiring unit 313 sequentially acquires brainwave signals measured through the ear canals of the user using the brainwave measuring device attached to the ears of the user. The brainwave measuring device is not limited to the earphone set 10. The brainwave signals may be brainwave signals indicating feature data (power vector at each frequency for a unit time) of ear canal brainwaves (In-Ear EEG) to be measured.

The learning unit 314 inputs the sequentially acquired brainwave signals into the learning data, which includes the brainwave signals and the sedation depth with respect to these brainwave signals, and performs supervised learning. For the brainwave signals, at least one of: a brainwave signal measured via a right ear, a brainwave signal measured via a left ear, and a differential signal of the left and right signals, for example, may be used. A signal calculated from the left and right signals, such as the differential signal, may be used.

The sedation depth is annotated to the brainwave signals, and may be attached to the brainwave signals as a teaching label for the level of RASS, for example. For the labeling, whether the sedation level is moderate or not can be predicted by separating the labels (attaching different labels) depending on, at least, whether the sedation level is less than moderate or not. As a result, the prediction accuracy and reliability can be improved at the moderate levels of sedation.

For the learning data, the brainwave signals having the teaching label of the sedation depth may be used as the learning data. For the learning model, the sparse modeling LASSO of the generalized linear model is used, for example, but this is just an example, and other learning models to solve classification problems and regression problems may be used instead.

The generating unit 315 receives learning data inputted by the learning unit 314 and generates a learned learning model as a prediction model. For example, in a case where a learned learning model based on the learning data is evaluated using test data, and the evaluated value is a threshold or more, the generating unit 315 may determine this learning model as a prediction model.

The output unit 316 outputs the prediction model generated by the generating unit 315. The output unit 316 is another information processing apparatus 50 that outputs a prediction model, such as a medical device, or a processing terminal, a personal computer, a smartphone, a tablet terminal, or the like, used by a user (e.g. physician). The other information processing apparatus 50 predicts the sedation depth of the user wearing the brainwave measuring device, using the prediction model to predict the sedation depth from the brainwave signals.

By the above processing, the information processing apparatus 30 can generate a sedation depth prediction model using the learning data measured for a subject user. By learning the learning data for many subject users, the information processing apparatus 30 can generate a more versatile sedation depth prediction model.

### Configuration Example of Processing Terminal

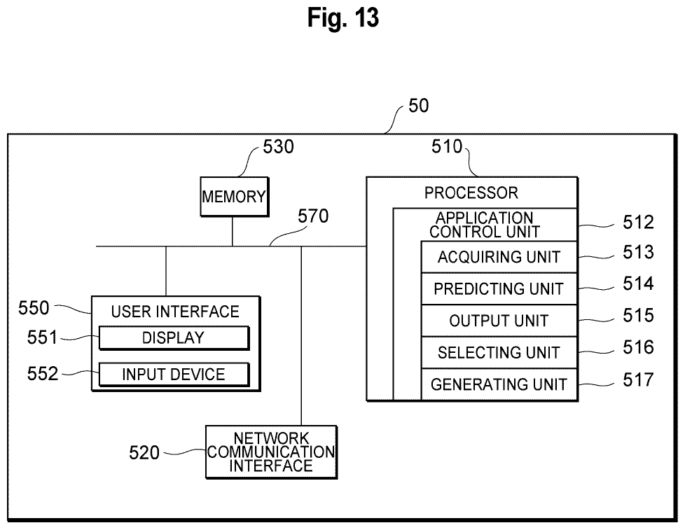

FIG. 13 is a block diagram depicting an example of the information processing apparatus 50 according to an embodiment. The information processing apparatus 50 includes a medical appliance, a portable terminal (e.g. smartphone), a computer, a table terminal, or the like, as mentioned above. The information processing apparatus 50 is also referred to as a processing terminal 50.

The processing terminal 50 includes one or a plurality of processors (e.g. CPUs) 510, one or a plurality of network communication interfaces 520, a memory 530, a user interface 550, and one or a plurality of communication buses 590 to interconnect these composing elements.

The user interface 550 includes a display 551 and an input device (e.g. keyboard and/or mouse, or a pointing device) 552. The user interface 550 may be a touch panel.

The memory 530 is, for example, DRAM, SRAM, DDR RAM or other high-speed rand access memory, such as a random access solid-state storage device. The memory 530 may also be one or a plurality of magnetic disk storage devices, optical disk storage devices, flash memory devices, or other non-volatile memories, such as non-volatile solid-state storage devices. The memory 530 may also be a computer-readable non-transitory recording medium in which programs are recorded.

Another example of the memory 530 is one or a plurality of storage devices which are remotely installed from the processor 510. In some cases, the memory 530 stores the following programs, modules, data structures or subsets thereof.

The one or plurality of processors 510 read programs from the memory 530 as required, and execute the programs. For example, the one or plurality of processors 510 may implement a control unit for applications (hereafter also called “application control unit”) 512 by executing the programs stored in the memory 530. The application control unit 512 is an application to process the brainwave signals, and includes an acquiring unit 513, a predicting unit 514, an output unit 515, a selecting unit 516, and a generating unit 517.

The acquiring unit 513 sequentially acquires the brainwave signals measured by the brainwave measuring devices attached to the ears of a predetermined user. For example, the acquiring unit 513 may acquire brainwave signals from the earphone set 10 via the network communication interface 520, or may acquire brainwave signals, after receiving predetermined processing, via the network communication interface 520. The predetermined processing includes, for example, frequency conversion, filter processing, difference calculation, and the like, performed on the brainwave signals. The acquiring unit 513 may acquire feature data (power vector at each frequency for a unit time) of the brainwave signals.

The acquiring unit 513 may perform predetermined processing on the brainwave signals which were acquired at a predetermined sampling rate. The predetermined processing includes bandpass filter processing in a predetermined range (e.g. 1 to 40 Hz), setting a time window for a predetermined time (e.g. 4 seconds), setting a predetermined moving time of time window (e.g. 1 second), and the like.

The predicting unit 514 predicts the sedation depth with respect to brainwave signals which are sequentially acquired by the acquiring unit 513, using the sequentially acquired brainwave signals and a learning model generated by learning the learning data, including the brainwave signals and sedation depth with respect to the brainwave signals. For example, the learning model may be a prediction model generated by the server 30, or a prediction model generated by the generating unit 517, or a prediction model customized by the predicting unit 514.

For example, the predicting unit 514 inputs the sequentially acquired brainwave signals to a prediction model generated by the server 30, and outputs the predicted values of the sedation depth. The sequentially acquired brainwaves signals may be brainwave signals after performing the predetermined processing is performed. The predicting unit 514 may also predict the sedation depth using a personal prediction model generated by the later mentioned generating unit 517.

The output unit 515 outputs the sedation depth information on the sedation depth predicted by the predicting unit 514. For example, the output unit 515 outputs the sedation depth information, including the predicted value of the sedation depth, to the display device (display 551 or an external display device). The output unit 515 may output an alarm to a sound output device (e.g. speaker) when the predicted value satisfies a predetermined condition triggering the alarm. For example, the predetermined condition triggering the alarm may be a predicted value exceeding a threshold.

By the above processing, the sedation depth can be appropriately predicted based on the brainwave signals, using a simple brainwave measuring device that can acquire brainwaves through the ears of the user, while decreasing a burden on the user in terms of attaching the device. In past research, sedation depth has been researched focusing on anesthetics, such as general anesthesia, but there is no technique to predict the depth of moderate sedation in endoscopic treatment. According to the technique of the present disclosure, the depth of moderate sedation can be appropriately predicted.

In the case of BIS, analysis is performed within a 1 minutes time range, hence a sudden change of sedation depth cannot be handled, but in the case of the technique of the present disclosure, the sedation depth is predicted based on the brainwave data in the most recent time window of a predetermined time (e.g. 4 seconds), hence prediction can be performed quickly, and a sudden change of sedation depth can be handled.

Further, conventional electrodes are often attached to the forehead for measurement, which means that ocular potential signals and myogenic potential signals tend to be included in the brainwave signals, and have a negative influence as noise. In the technique of the present disclosure, on the other hand, the wearable earphone set 10 of a simple electro-encephalograph can easily be worn, just like regular earphones, and brainwave signals can be easily acquired via the ear canal electrodes. Therefore burden on the user when the device is attached can be decreased.

Since the brainwave measuring device is included in the earphone set 10, as mentioned above, regular earphones may be used and sound may be outputted from the earphone set 10. By the earphone set 10, including both functions of the earphones to measure brainwaves and of the earphones to output sound, the information processing apparatus 50 may perform the following processing.

The selecting unit 516 selects sound data based on the predicted sedation depth. For example, in a case where the predicted value of the sedation depth becomes below a threshold, the selecting unit 516 may select one sound out of a playlist including one or a plurality of sounds that generally regarded as having a high sedation effect (music, binaural beats). For example, it is known that by sending acoustic stimulations having mutually different frequencies to the brain through the left and right ears (binaural beats), brainwaves in differential frequency bands can be reinforced (Schmid, W. et al. Brainwave entrainment to minimise sedative drug doses in paediatric surgery: a randomised controlled trial. Br. J. Anaesth. 125, 330 335 (2020 )).

In this case, the output unit 515 may output the sound data selected by the selecting unit 516 to the earphone set 10. For example, the output unit 515 outputs the selected sound data to the earphones 100 of both ears, or to an earphone of one ear, 100L or right.

If the above processing is used, the brainwave measuring device can support the sedative effect utilizing the function to output a sound.

The sound data to be outputted may include sound data specified for the subject user of the brainwave measurement. For example, the subject user of the brainwave measurement selects healing music to calm their mind, favorite music, and the like in advance, and a playlist thereof is created. The selecting unit 516 selects sound data out of the private playlist of the user.

By the above processing, sound data can be selected from the playlist appropriate for the subject user of the brainwave measurement, and the sedative effect can be supported by the sound data liked by the user.

The acquiring unit 513 may sequentially acquire brainwave signals in the case of administering anesthetic to the user. In this case, the output unit 515 may output information on the dosage of the anesthetic based on the sedation depth predicted by the predicting unit 514.

For example, the information on the dosage of the anesthetic may include the dosage to be administered to the user, or a difference value between the current dosage and the dosage to be administered. For example, the predicting unit 514 may specify the dosage of the agent to be administered to the user in accordance with the predicted sedation depth, by referring to a correspondence table between the sedation depth and the dosage to be administered. The correspondence table may be stored in the memory 530, or the like, for each agent. If the information processing apparatus 50 can acquire the current dosage being administered to the user from another device, the predicting unit 514 may specify the difference value between the current dosage and the dosage to be administered.

By the above processing, the dosage to be administered to the user next, the timing of administering, and the like, can be outputted to the display device or the like, in accordance with the predicted level of sedation depth. As a result, the physician or the like can determine the dosage of the agent and the timing of administering based on objective data.

The generating unit 517 executes the generation of the learning model based on the brainwave signals acquired from the subject user of the brainwave measurement during a predetermined time, and the sedation depth of the user with respect to the brainwave signals. For example, the generating unit 517 may generate a prediction model appropriate for the user by inputting the learning data including the brainwave signals acquired during the predetermined time, and a teaching label, annotated to the brainwave signals, into the learning model.

In the case where the generating unit 517 generates a prediction model, the predicting unit 514 may predict the sedation depth of the user by inputting the brainwave signals, which are sequentially acquired after the predetermined time has elapsed, to the prediction model. For example, by using the prediction model generated for each user, the predicting unit 514 can predict the sedation depth in accordance with the features of the brainwave data of the user.

In a case where the anesthetic (an example of an agent) is administered within a predetermined time, the generating unit 517 may determine the level of the awakened state in the sedation depth of the user, based on the brainwave signals which are acquired from the user before this predetermined time and also before the anesthetic is administered. For example, the power of the brainwave signals in the awakened state may be different depending on the user, hence the generating unit 517 may set a standard of the brainwave signals in the awakened state (teaching label in the awakened state) of this user, using the brainwave signals during a predetermined time before the anesthetic is administered.

For example, the generating unit 517 may set a standard of the awakened state using the brainwave signals for a first predetermined time (e.g. 3 minutes), then may generate a prediction model to predict the sedation depth of this user using the learning data generated by performing annotation on the brainwave signals for a second predetermined time (e.g. 3 minutes) after the anesthetic was administered after the first predetermined time has elapsed.

By the above processing, the prediction model, to predict the sedation depth from the brainwave signals, can be generated in accordance with the brainwave state of each user. As a result, the sedation depth can be more appropriately predicted in accordance with the features of the brainwave state of the subject user of the brainwave measurement.

The predicting unit 514 may remove brainwave signals that are equal to or more thana threshold, out of the sequentially acquired brainwave signals. For example, to remove brainwave signals that mix with noise, the predicting unit 514 or the acquiring unit 513 may remove the brainwave signals that are equal to or more than the threshold (e.g. amplitude value) from the values to be inputted to the prediction model. In the above mentioned experiment, 50 μV was used for the threshold.

By the above processing, brainwave signals that mix with noise entered during treatment by use of an electric scalpel, or the like, can be removed, and reliability of the predicted value of the sedation depth can be improved.

In the case where body movement and vital information can be acquired, as indicated in FIG. 6, the application control unit 512 may store this information in the memory 530 linking it with the brainwave signals. Thereby the relationship of the brainwave signals, sedation depth, and vital information can be examined.

The above describes an example of generating a personal prediction model using a first predetermined time to determine a standard of the awakened state, and a second predetermined time to generate the learning data, but a generalized prediction model may be generated in a case where a number of subject users of brainwave measurement is high. For example, a generalized standard of the awakened state may be set using the brainwave signals of each user in the first predetermined time, then a generalized prediction model can be generated using this standard of the awakened state and learning data generated in the second predetermined time. In the case of using the generalized prediction model (e.g. prediction model generated by the server 30), a time for learning is not required, hence the prediction model can be used as soon as treatment starts.

By storing information on the administered agent and the brainwave signals linked with each other in advance, the application control unit 512 can predict the sedation depth for each agent. For example, the application control unit 512 can predict how the sedation depth will change in the future once the type of agent and dosage thereof are known. Specifically, the application control unit 512 links a prediction model to each agent in advance, and once an agent to be administered is determined, the predicting unit 514 performs prediction processing using the prediction model inked to the agent.

Each information outputted to the display device or sound output device is as follows, for example.
Predicted value of the sedation depth in 0 to 100 range, which is calculated in real-time (prediction of sedation depth is updated about every 0.1 seconds based on data of brainwaves for most recent 4 seconds).
Alert sound based on a specific threshold, and visual output of predicted value in real-time and log data of predicted value. Thereby the sedation tendency can be recognized in real-time and over a period of time.
The noise level in real-time. Thereby the user can evaluate the reliability of the current predicted value (e.g. high noise state is indicated by a gray display while an electric scalpel is in use).

### Operation

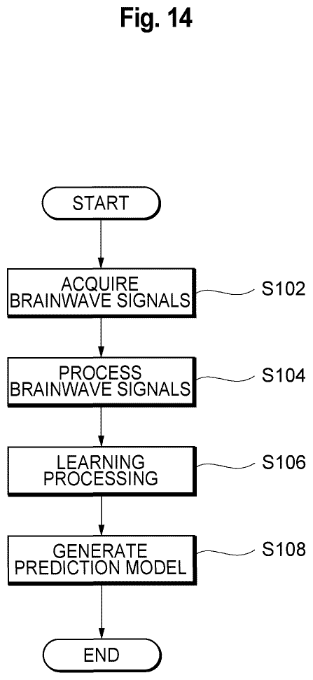

Operation according to an embodiment will be described next. FIG. 14 is a flow chart depicting an example of the prediction model generating processing by the server 30 according to the embodiment. In the example in FIG. 14, the processing, when the server 30 generates a prediction model to predict the sedation depth from the brainwave signals, will be described. The processing in FIG. 14 can also be performed by the application control unit 512 (e.g. generating unit 517) of the processing terminal 50.

In step S102, the acquiring unit 313 sequentially acquires the brainwave signals from the brainwave measuring device attached to the ears of the user. For example, the acquiring unit 313 sequentially acquires the brainwave signals measured via the bioelectrodes disposed in the ear chips of the earphones.

In step S104, the acquiring unit 313 or the learning unit 314 performs processing on the acquired brainwave signals. The processing includes, for example, difference calculation of the left and right brainwave signals, frequency conversion, power value calculation at each frequency, and filter processing, performed on the brainwave signals.

In step S106, the learning unit 314 performs machine learning using learning data which includes sequentially acquired brainwave signals after executing the processing, and the sedation depth (teaching label) of these brainwave signals.

In step S108, the generating unit 315 generates the learned model, which the learning unit 314 learned using the learning data, as the prediction model.

By the above processing, the sedation depth prediction model can be generated in advance using the learning data measured for the subject user.

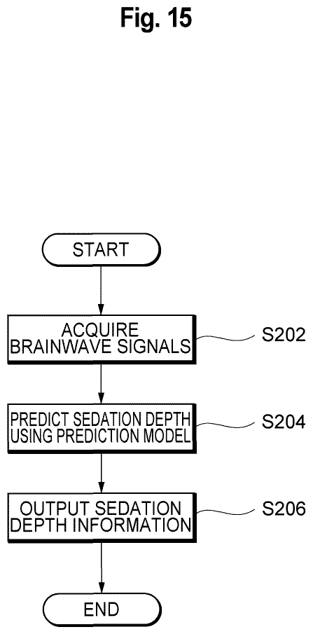

FIG. 15 is a flow chart depicting an example of prediction processing of the sedation depth by the processing terminal 50 according to the embodiment. In the example in FIG. 15, processing, when the processing terminal 50 predicts the sedation depth from the brainwave signals, will be described.

In step S202 in FIG. 15, the acquiring unit 513 sequentially acquires the brainwave signals from the brainwave measuring device attached to the ears of the user. The brainwave signals may refer to the brainwave signals generated after executing the predetermined processing (e.g. frequency calculation) mentioned above.

In step S204, the predicting unit 514 predicts the sedation depth with respect to the sequentially acquired brainwave signals, using the sequentially acquired brainwave signals and a learning model (prediction model) which has learned learning data including brainwave signals and a sedation depth with respect to the brainwave signals.

In step S206, the output unit 515 outputs the sedation depth information on the sedation depth predicted by the predicting unit 514. For example, the output unit 515 outputs the sedation depth information to the display device.

By the above processing, the sedation depth can be predicted based on the brainwave signals, using a simple brainwave measuring device which can acquire brainwaves through the ears, reducing burden on the user when the device is attached. Further, according to the technique of the present disclosure, the sedation depth can be predicted and presented virtually in real-time, hence even a sudden change of sedation depth can be handled.

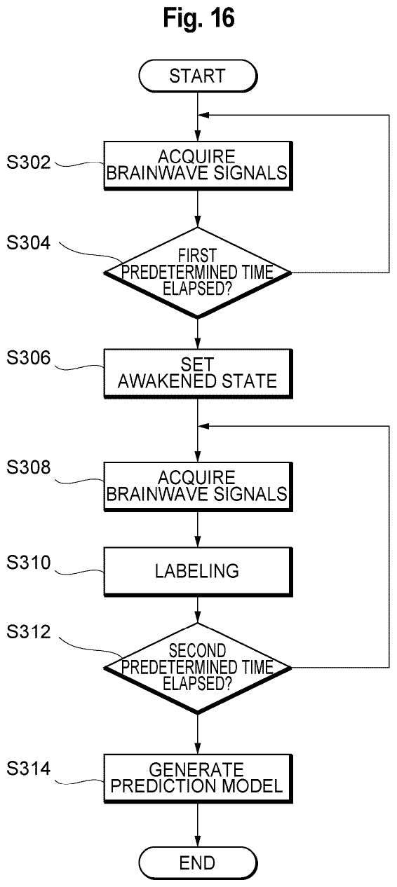

FIG. 16 is a flow chart depicting an example of learning model generating processing by the processing terminal 50 according to the embodiment. The processing in FIG. 16 can be executed by the application control unit 512 of the processing terminal 50, particularly by the acquiring unit 513 and the generating unit 517.

In step S302 in FIG. 16, the acquiring unit 513 sequentially acquires brainwave signals measured via the bioelectrodes contacting the ear canals of a predetermined user. For example, the acquiring unit 513 sequentially acquires the brainwave signals measured via the bioelectrodes disposed in the ear chips of the earphones. The brainwave signals may refer to the brainwave signals generated after executing predetermined processing (e.g. frequency calculation) mentioned above.

In step S304, the application control unit 512 determines whether a first predetermined time has elapsed since the start of acquiring the brainwave signals. The first predetermined time is 3 minutes, for example. Processing advances to step S306 if the first predetermined time has elapsed (step S304: YES), or processing returns to step S302 if the first predetermined time has not elapsed (step S304: NO).

In step S306, the generating unit 517 sets a standard value of the brainwave signals in the awakened state of the subject user of the brainwave measurement, using the brainwave signals measured in the first predetermined time. For example, the generating unit 517 assigns a teaching label indicating the awakened state to the average value of the brainwave signals in the awakened state.

In step S308, the agent is administered when the first predetermined time elapsed, then the acquiring unit 513 sequentially acquires brainwave signals measured via the bioelectrodes contacting the ear canals of the predetermined user.

In step S310, the generating unit 517 performs labeling by assigning the sedation depth level set by an operator (e.g. physician) to the brainwave signal at that timing.

In step S312, the application control unit 512 determines whether a second predetermined time has elapsed since administering the agent to the subject user of the brainwave measurement. The second predetermined time is 3 minutes, for example. Processing advances to step S314 if the second predetermined time has elapsed (step S310: YES), or processing returns to step S308 if the second predetermined time has not elapsed (step S310: NO).

In step S314, the generating unit 517 performs the supervised learning using the brainwave signals (learning data) annotated in the second predetermined time, and generates the prediction model for the user.

By the above processing, the prediction model, to predict the sedation depth from the brainwave signals, can be generated in accordance with the brainwave state of each subject user. As a result, the sedation state can be predicted more appropriately in accordance with the features of the brainwave state of the subject user of the brainwave measurement.

### Screen Example

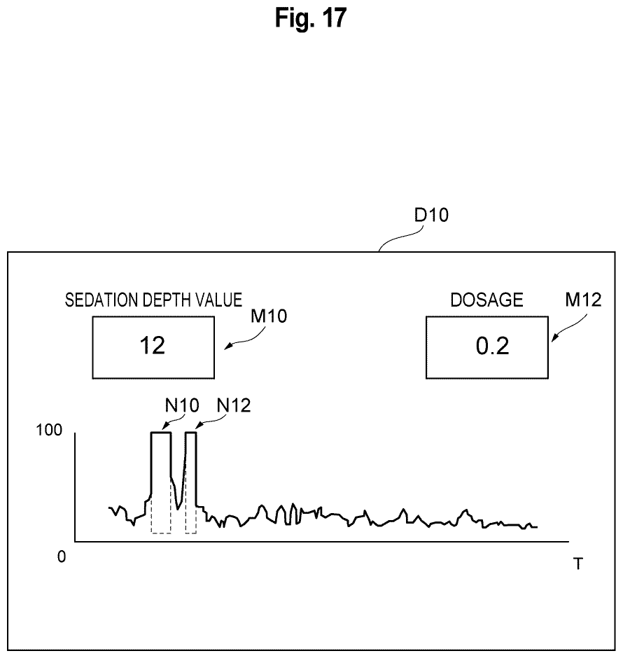

FIG. 17 is a diagram indicating an example of a display screen according to the embodiment. A screen D10 indicated in FIG. 17 includes a time series data of the predicted values of the sedation depth of the technique of this disclosure, a sedation depth value (predicted value) M10, and a current dosage M12 of agent.

In the example in FIG. 17, predicted values of the sedation depth, predicted using the prediction model of the technique in this disclosure, are displayed in a time series. In this time series data, it is preferable that noise N10 and N12, which entered during treatment with an electric scalpel, for example, can visually be identified from regular predicted values in the display. For example, the noise may be recognized at a glance by surrounding the noise with a frame or by changing the color of the noise, as indicated in FIG. 17.

In the sedation depth value M10, a latest predicted value of the sedation depth is displayed. The sedation depth is deeper as the sedation depth value is lower, and is closer to the awakened state as the sedation depth value is higher. The sedation depth value may be normalized to a value in the 0 to 100 range, for example. Here if the sedation depth value is equal to or more thana threshold indicating an awakened state, or if the sedation depth value becomes equal to or less thana threshold, indicating an excessively deep sedation state, an alarm may be outputted or the color of the sedation depth value may be changed.

The dosage M12 indicates a current dosage of the agent. The dosage M12 may also be a dosage of the agent to be administered next, which is predicted based on the predicted value of the sedation depth. The dosage M12 may not always be an essential display item.

### Modifications

The embodiments and examples described above are merely examples to describe a technique of the present disclosure, and are not intended to limit the technique of the present disclosure to only these embodiments and examples. The technique of the present disclosure can be modified in various ways within a scope not departing from the spirit thereof. For example, each processing step in FIGS. 14 to 16 may be swapped or omitted if necessary, or another processing step may be inserted. The brainwave signals may be regarded as the brainwave data.

### Modification 1

In the above example, an anesthetic was described as an example of the agent, but the technique of the present disclosure is also applicable to other agents that result in a sedation effect, such as sleeping pills and tranquilizers. For example, when the user takes sleeping pills or tranquilizers, the relationship of the brainwave signals of the user when the agent is taken and the sedation depth of the user at this time can be learned. In this case, the sedation state of the user can be supported by outputting sound data based on the sedation depth, as mentioned above. Thereby the dosage of the sleeping pills or tranquilizers taken by the user can be reduced.

For example, the above mentioned brainwave signal acquiring processing, sedation depth predicting processing, output processing, and the like, may be executed on the processing terminal 50 by pairing the earphone set 10 and the processing terminal 50 via short range wireless communication or the like.

### REFERENCE SIGNS LIST

1 Information processing system

10 Earphone set

30, 50 Information processing apparatus

100 Earphone

104 Nozzle

106 Ear chip (elastic electrode)

310 Processor

312 Brainwave control unit

313 Acquiring unit

314 Learning unit

315 Generating unit

316 Output unit

330 Memory

510 Processor

512 Application control unit

513 Acquiring unit

514 Predicting unit

515 Output unit

516 Selecting unit

517 Generating unit

530 Memory

550 User interface

1. An information processing method, which is executed by an information processing apparatus, the method comprising executing:
sequentially acquiring brainwave signals from a brainwave measuring device attached to ears of a user;
predicting sedation depth with respect to the sequentially acquired brainwave signals by using the sequentially acquired brainwave signals and a learning model which has learned learning data including brainwave signals and sedation depth with respect to the brainwave signals; and
outputting sedation depth information on the predicted sedation depth.

2. The information processing method according to claim 1, wherein the brainwave measuring device is included in an earphone set,
the method further comprising executing:
selecting sound data, based on the predicted sedation depth; and
outputting the selected sound data to the earphone set.

3. The information processing method according to claim 2, wherein the sound data includes sound data specified in accordance with the user.

4. The information processing method according to claim 1, wherein the acquiring includes sequentially acquiring brainwave signals in a case where anesthetic is administered to the user, and
the outputting includes outputting information on dosage of the anesthetic based on the predicted sedation depth.

5. The information processing method according to claim 4, wherein the information on dosage of the anesthetic includes dosage to be administered to the user, or a difference value between current dosage and the dosage to be administered.

6. The information processing method according to claim 1, wherein
the learning model is generated based on brainwave signals acquired from the user for a predetermined time, and sedation depth of the user with respect to the brainwave signals, and
the predicting includes predicting the sedation depth of the user by inputting brainwave signals, sequentially acquired after the predetermined time elapsed, to the learning model.

7. The information processing method according to claim 6, wherein
in a case where anesthetic is administered during the predetermined time, the generating includes determining a level of awakened state in the sedation depth of the user, based on brainwave signals which are acquired from the user before start of the predetermined time and before the anesthetic is administered.

8. The information processing method according to claim 1, wherein
the predicting includes removing brainwave signals that are equal to or more thana threshold out of the sequentially acquired brainwave signals.

9. The information processing method according to claim 1, wherein
labels of the sedation depth included in the learning data are different depending on at least whether or not the sedation depth is less than, or equal to or more than a moderate sedation.

10. A program for causing an information processing apparatus to execute:
sequentially acquiring brainwave signals from a brainwave measuring device attached to ears of a user;
predicting sedation depth with respect to the sequentially acquired brainwave signals by using the sequentially acquired brainwave signals and a learning model which has learned learning data including brainwave signals and sedation depth with respect to the brainwave signals; and
outputting sedation depth information on the predicted sedation depth.

11. An information processing apparatus including a processor,
the processor executing:
sequentially acquiring brainwave signals from a brainwave measuring device attached to ears of a user;
predicting sedation depth with respect to the sequentially acquired brainwave signals by using the sequentially acquired brainwave signals, and a learning model which has learned learning data including brainwave signals and sedation depth with respect to the brainwave signals; and
outputting sedation depth information on the predicted sedation depth.
